(19) United States
(12) Patent Application Publication
(10) Pub. No.: US 2022/0208540 A1
(43) Pub. Date: Jun. 30, 2022

(54) System for Identifying Structures of Molecular Compounds from Mass Spectrometry Data

(71) Applicant: Carnegie Mellon University, Pittsburgh, PA (US)

(72) Inventors: Bahar Behsaz, Pasadena, CA (US); Liu Cao, Pittsburgh, PA (US); Mustafa Guler, Pittsburgh, PA (US); Yi-Yuan Lee, Ithaca, NY (US); Hosein Mohimani, Pittsburgh, PA (US)

(21) Appl. No.: 17/554,690

(22) Filed: Dec. 17, 2021

Related U.S. Application Data
(60) Provisional application No. 63/126,591, filed on Dec. 17, 2020.

(51) Int. Cl. H01J 49/26; G06N 20/00; G01N 33/68; C07K 1/04; G16B 40/10

(52) U.S. Cl. CPC H01J 49/26; G06N 20/00; G16B 40/10; C07K 1/047; G01N 33/6818

(57) ABSTRACT

A method and system is for searching a database to identify structures of molecular compounds from mass spectrometry data. Operations of the method and system include receiving a query for a target molecular structure in the database, the query representing a query spectrum; accessing a machine learning model trained with molecule-spectrum pairs; inputting the query spectrum into the machine learning model; generating, from the machine learning model, a score for each of one or more molecular structures, each score representing a probability that a molecular structure corresponds to the query spectrum; selecting, based on each of the scores, a small molecule; and outputting, on a user interface, a representation of the small molecule.

### CLAIM OF PRIORITY

This application claims priority under 35 U. S.C. § 119(e) to U.S. Patent Application Ser. No. 63/126,591, filed on Dec. 17, 2020, the contents of which are hereby incorporated by reference.

### GOVERNMENT SUPPORT CLAUSE

This invention was made with United States government support under grant number DP2GM137413, awarded by the National Institutes of Health and under grant DE-SC0021340, awarded by the Department of Energy. The U.S. Government has certain rights in the invention.

### TECHNICAL FIELD

This disclosure relates to drug discovery. More specifically, this disclosure relates to machine learning models configured for determining molecular structures of compounds based on the mass spectra of those compounds.

### BACKGROUND

Identification of small molecule compounds, including natural products and other compounds, is a process used for drug discovery. Recent advances in mass spectrometry have enabled the collection of tandem mass spectra (MS/MS) of small molecules from hundreds of thousands of environments. To identify which molecules are present in a sample, one can search mass spectra collected from the sample against millions of molecular structures in databases of such fragments, which is a spectral library search. This is a challenging task as currently it is not clear how small molecules are fragmented in mass spectrometry. The existing approaches, including in-silico search of small molecule structure databases, use the domain knowledge from chemistry to predict fragmentation of molecules, however these rule-based methods fail to explain many of the peaks in mass spectra of small molecules.

### SUMMARY

This disclosure describes systems and methods for determining a structure of compounds from mass spectrometry data. The system is configured to determine a two-dimensional (2D) structure that indicates the bond structure between molecules of a compound by analysis and classification of mass spectra of the compound. Generally, samples of natural products or other materials are received. The system is configured to generate genomics data from the samples of those natural products or other materials. The genomics data indicate genetic data for which microbes are in the sample and the potential for generating therapeutic small-molecule compounds (e.g., drugs). From the same samples, metabolomics data are obtained. This includes performing mass spectrometry of the samples to determine the chemistry of the metabolites in the samples.

The systems and methods described in this specification are configured to relate the genomics data and the metabolomics spectra data from the samples. From this relationship, machine learning models are trained to predict the natural product of the sample and determine which microbes present in the sample are responsible for generating the natural product. The data processing pipeline can be as follows. Microbial samples are obtained. From the microbial samples, the system performs multi-omics sequencing to generate the genomics data as previously described. The system is configured for data mining to develop the relationship between the genomics data and the metabolomics data. From the relationship, one or more compounds are identified (e.g., small molecule compounds). The system can then purify the identified compounds. The purified compounds are then tested for different bacteria (e.g., simulating bacterial infections). The final result is that new chemical entities and their efficacies are identified for use as drugs.

The one or more advantages previously described can be enabled by one or more embodiments. The processes described in this specification enable drug discovery using mass spectra. The accuracy and scalability of determining the structure of the compounds is improved relative to conventional methods for drug discovery from natural products, such as those subsequently described, and for structure analysis of compounds. For example, the traditional approaches are labor intensive, expensive, and inaccurate as they tend to miss viable drug candidates. The methods presented here are eight times more sensitive in detection of small molecules from mass spectra.

Existing models for metabolomics are inaccurate because there is no good model to predict a molecular structure from a mass spectra. Currently, a molecular structure is divided into fragments, and mass spectra of these fragments are measured. This disclosure describes a model is trained with fragments from mass spectrometry to predict the chemical structure.

The data processing system is configured to determine which known small molecules are present or absent in a specific sample (e.g., a microbial sample). For example, the data processing system is configured to identify small molecule biomarkers in plasma/oral/urinal/fecal/tissue samples from a patient for disease diagnosis or prognosis. Epidemiologists are interested in identifying small molecule disease risk factors from diet and the environment. Ecologists are interested in characterizing the molecules produced by microbes in various microbial communities. Natural product researchers are trained to identify all known molecules in their sample, clearing the path towards the discovery of novel antimicrobial or antitumor molecules.

Advances in high-throughput mass spectrometry have enabled collection of billions of mass spectra from hundreds of thousands of host-oriented/environmental samples. A mass spectrum is the fingerprint of a small molecule, which can be represented by a set of mass peaks. To identify small molecules in a sample with tens of thousands of spectra, one can either de novo predict small molecule structure corresponding to mass spectra, search these mass spectra against tens of thousands of reference spectra in spectral libraries, or search these mass spectra against millions of molecular structures in small molecule databases. De novo prediction can potentially identify both known and novel small molecules. However, de novo prediction is rarely used in practice because of an intrinsic complexity of small molecule structures and a low signal-to-noise ratio of mass spectral data. While spectral library search is generally the most reliable mass spectral annotation method, current reference spectral libraries are limited to tens of thousands of molecules, and the majority of known small molecules are not represented in any reference spectral library. Furthermore, associating mass spectra of all known small molecules individually is expensive and time-consuming. The most frequently used strategy for small molecule identification is in-silico search of small molecule structure databases, this approach enables small molecule identification in known databases. Moreover, in-silico database search also applies to discovery of novel small molecules through genome mining.

The majority of in-silico database search methods are rule-based models that incorporate domain knowledge, such as bond types, hydrogen rearrangement, and dissociation energy to predict fragmentations of small molecules and score small molecule-spectrum pairs. However, due to the complex rules involved in small molecule fragmentation, these methods are computationally inefficient for heavy small molecules. Moreover, these predictions, which are based on expert knowledge, do not explain many of the peaks in mass spectra.

As previously described, the efficiency and accuracy of small molecule identification is achieved by the data processing system by (i) designing an efficient algorithm to generate mass spectrometry fragmentations and (ii) developing a probabilistic model to identify small molecules from their mass spectra.

The methods described herein enable a lower false discovery rate (FDR) having an eight-fold improvement compared to existing methods. For example, on a subset of the GNPS repository with known genomes, the data processing system described in this specification correctly linked 20 known and three novel biosynthetic gene clusters to their molecular products.

The method and system described herein each provide scalable, efficient and accurate in-silico predictions and identification of varying classes of compounds with an expanded range of up to 5000 Daltons (Da) of molecular weight. Furthermore, the systems described herein are more efficient than existing methods in terms of run-time and memory consumption. For example, run-time can be reduced by a factor of 60 and memory consumption can be reduced by a factor of 1000.

The method and system for searching databases of mass spectrometry (MS) data, improve the efficiency and accuracy of compound identification from MS data, such as from MS/MS fragmentation data. In some embodiments, the present invention can identify compounds in complex mixtures. The system includes an efficient algorithm to construct fragmentation graphs of compounds, including hypothetical molecules, enabling in-silico database search for molecules up to 5000 Da. The data processing system is configured to construct a metabolite graph for a compound structure and then generate a fragmentation graph from the metabolite graph.

In some implementations, the metabolite graph for hypothetical molecules is made by applying hypothetical modifications of known molecules.

In some implementations, a machine learning model is used by the data processing system to match compounds with their mass spectra and improves the accuracy of the database searching. In some implementations, the machine learning models can include a probabilistic learning model to match compounds with their mass spectra and improve the accuracy of database searching.

The data processing system is configured to determine molecules predicted as the products of biosynthetic gene clusters, including non-ribosomal peptide biosynthetic gene clusters, ribosomally synthesized and post-translationally modified peptide biosynthetic gene clusters, polyketide biosynthetic gene clusters, carbohydrate biosynthetic gene clusters, aminoglycoside biosynthetic gene clusters, and polysaccharide biosynthetic gene clusters.

Additional examples of compounds identified by the data processing system include small molecules, small molecule natural products (including from the class of non-ribosomal peptides, polyketides, ribosomally synthesized and post-translationally modified peptides, carbohydrates, aminoglycosides, and polysaccharides.

In a general aspect, a computer-implemented process is for searching a database to identify structures of molecular compounds from mass spectrometry data. The operations of the process include receiving a query for a target molecular structure in the database, the query representing a query spectrum; accessing a machine learning model trained with molecule-spectrum pairs, wherein a molecule-spectrum pair of the molecule-spectrum pairs comprises structure data representing two-dimensional molecular structures of small molecules, the structure data being associated with spectrum data representing a mass spectrum that is generated from the small molecules represented in the structure data; inputting the query spectrum into the machine learning model; generating, from the machine learning model, a score for each of one or more molecular structures, each score representing a probability that a molecular structure corresponds to the query spectrum; selecting, based on each of the scores, a small molecule; and outputting, on a user interface, a representation of the small molecule.

In some implementations, the machine learning model enables a reduction in computing memory and a decrease in latency of returning the representation of the small molecule in response to receiving the query spectrum, the reduction being relative to a rule-based model using at least one of bond type data, hydrogen rearrangement data, and dissociation energy data for searching the database.

In some implementations, the operations further include training the machine learning model. Training the machine learning model includes generating, for a given small molecule of the structure data, a fragmentation graph, the fragmentation graph representing one or more fragments of the small molecule, the one or more fragments corresponding to mass spectrum peaks of the small molecule; and assigning each of the one or more fragments of the fragmentation graph, a bond type and a log rank.

In some implementations, the bond type represents chemical bonds that are disconnected in a parent fragment to produce a fragment.

In some implementations, the log rank represents an intensity of a mass peak corresponding to the fragment.

In some implementations, the fragmentation graph is generated by performing operations comprising: cutting, for the small molecule, N—C, O—C, and C—C bonds of the small molecule to generate sub-fragments to form a metabolite graph; generating depth one fragments by searching the metabolite graph for each cut bond; determining intersections between each depth one fragment and a corresponding parent fragment; forming depth two fragments based on the intersection; and connecting the metabolite graph to each depth one node and each depth two node to a corresponding depth one node.

In some implementations, assigning the fragmentation graph a log rank comprises: accessing mass spectra data including a plurality of mass spectra; for each mass spectra of the plurality: determining an intensity of each mass peak of the mass spectrum; and assigning a value for the log rank, wherein the value represents a higher rank as a function of a number of instances of the fragment in the fragmentation graph.

In some implementations, the operations include identifying a drug based on the identified small molecule.

In some implementations, the operations include evaluating an accuracy of the probability that a molecular structure corresponds to the query spectrum by applying a constrained graph variational auto-encoder.

In some implementations, the operations include receiving data representing at least one biosynthetic gene cluster including a non-ribosomal peptide biosynthetic gene cluster, a ribosomally synthesized and post-translationally modified biosynthetic gene cluster, a polyketide biosynthetic gene cluster, a carbohydrate gene cluster, a polysaccharide gene cluster, or an aminoglycoside gene cluster; generating a predicted mass spectrum associated with the at least one biosynthetic gene cluster; and training the machine learning model using the predicted mass spectrum and the at least one biosynthetic gene cluster.

In some implementations, a p-value associated with the molecular structure and the query spectrum is determined based on a Markov Chain Monte Carlo model.

In a general aspect, a system for searching a database to identify structures of molecular compounds from mass spectrometry data includes at least one processor; and a memory storing instructions that, when executed by the at least one processor, cause the at least one processor to perform operations previously described.

In a general aspect, one or more non-transitory computer readable media store instructions for searching a database to identify structures of molecular compounds from mass spectrometry data, the instructions, when executed by at least one processor, being configured to cause the at least one processor to perform the operations previously described.

The details of one or more implementations are set forth in the accompanying drawings and the description below. Other features and advantages will be apparent from the description and drawings, and from the claims.

### BRIEF DESCRIPTION OF DRAWINGS

FIG. 1A shows an example system for identifying the structures of molecular compounds from mass spectrometry data.

FIG. 1B shows an example process for identifying the structures of molecular compounds from mass spectrometry data using the system of FIG. 1A, in a case where a probabilistic learning model is used.

FIG. 1C shows an example process for identifying the structures of molecular compounds from mass spectrometry data using the system of FIG. 1A, in a case where a message passing neural network model is used.

FIG. 2 shows an example process for executing trained a machine learning model to identify the structures of molecular compounds from mass spectrometry data.

FIG. 3A shows an example process for training and executing a machine learning model to identify the structures of molecular compounds from mass spectrometry data and sequenced genomes.

FIG. 3B shows an example of a machine learning model for predicting the structure of natural products from genome sequence in case of ribosomally synthesized and post-translationally modified peptides.

FIG. 3C shows an example process for determining a molecular structure based on genomic data in case of Ribosomally Synthesized and post-translationally modified peptides.

FIG. 3D shows an example process for mapping structures of molecules to spectra data.

FIG. 4 shows an example process for identifying a natural product drug from microbial samples.

FIG. 5 shows a flow diagram representing a process for identifying the structures of molecular compounds from mass spectrometry data.

Like reference symbols in the various drawings indicate like elements.

### DETAILED DESCRIPTION

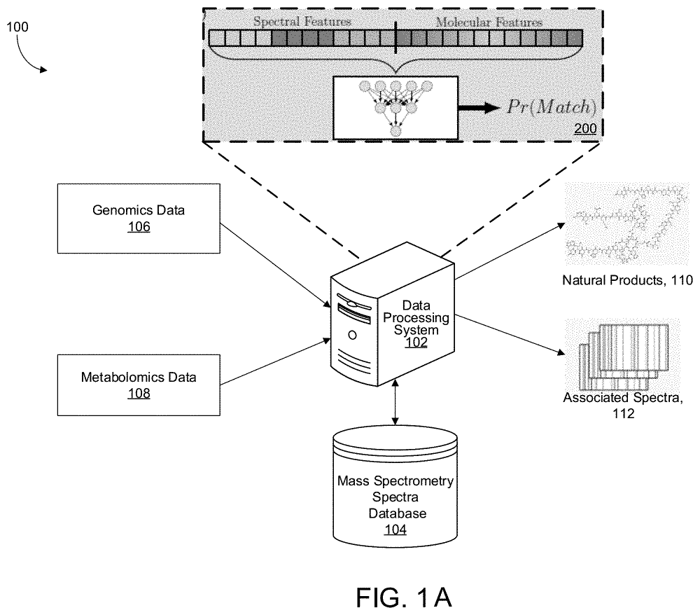

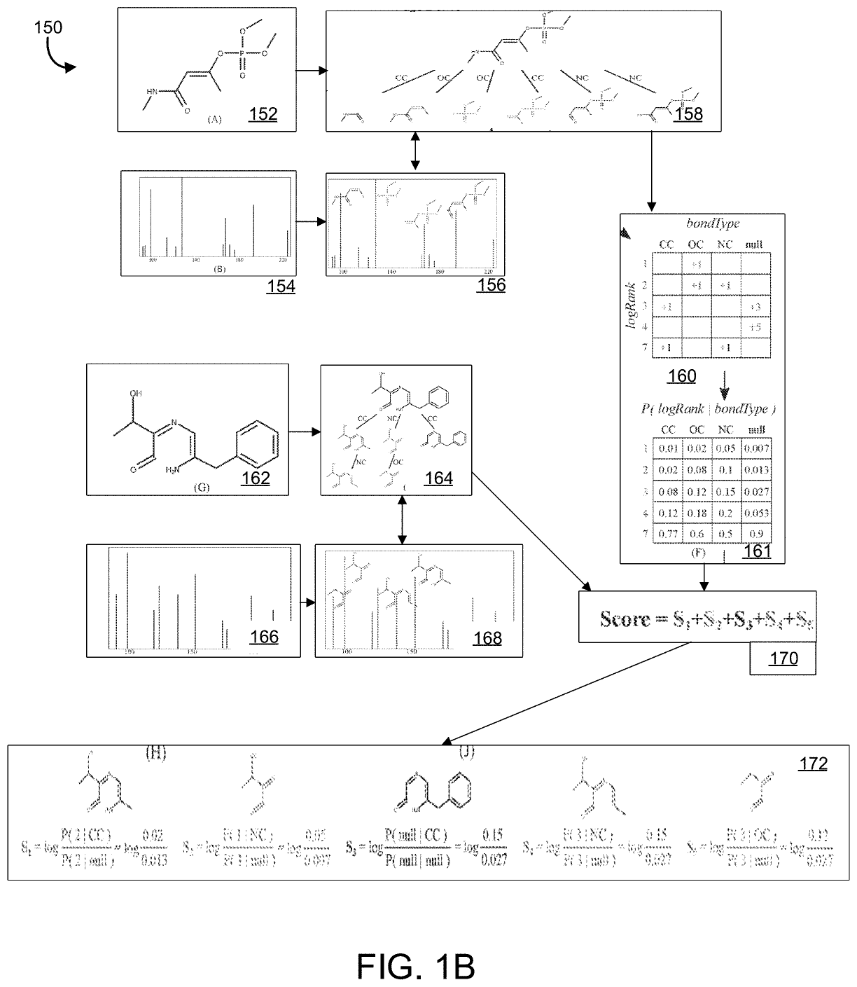

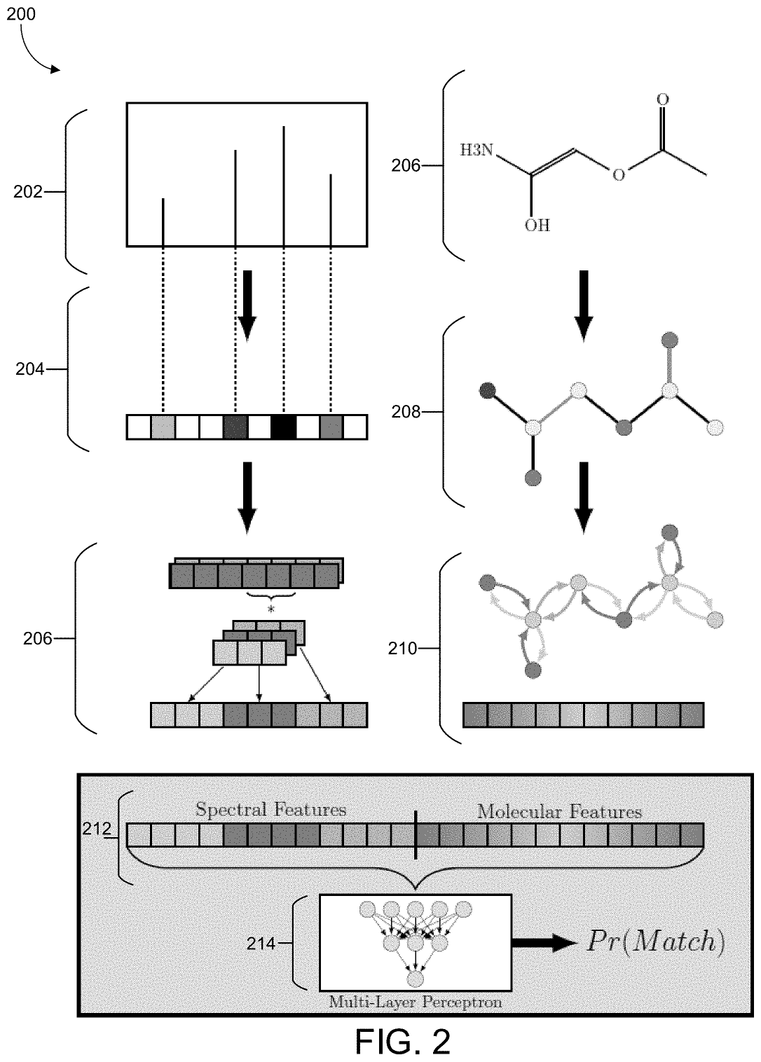

FIG. 1A shows an example environment 100 of a data processing system 102 for identifying the structures of molecular compounds from mass spectrometry data. The data processing system 102 includes a computing system that is configured to execute one or more machine learning models. The machine learning models receive genomics data 106 and metabolomics data 108. In this specification, genomics data 106 includes gene sequences. In this specification, metabolomics data 108 include data describing the metabolome. The metabolome includes a set of small-molecule (<5 kDa) metabolites, such as metabolic intermediates, hormones and other signaling molecules, and secondary metabolites, that are found within a biological sample, such as a single organism (e.g., a microbe). The data processing system 102 receives spectra data from a mass spectrometry spectra database 104 (database 104). The data processing system 102 processes the genomics data 106 and the metabolomics data 108 to identify natural products from spectra of the metabolomics data and pair these natural products with their respective spectra. The data processing system 102 is configured to output one or more identified natural products data 110 from the received samples (e.g., in the metabolomics data 108) associated with a given spectra. The natural products data 110 include a representation of small molecule structures (e.g., two dimensional structures) represented by the spectra of the samples. In some implementations, the output includes a ranked list of natural products in the natural products data 110. The data processing system 102 generally outputs data linking or associating the identified natural products of the natural products data 110 with each spectra of the spectra data 112. The spectra data 112 include results of performing mass spectrometry on a sample. In some implementations, the spectra data 112 are included in the metabolomics data 108. In some implementations, the spectra data 112 are generated as part of the processes described herein.

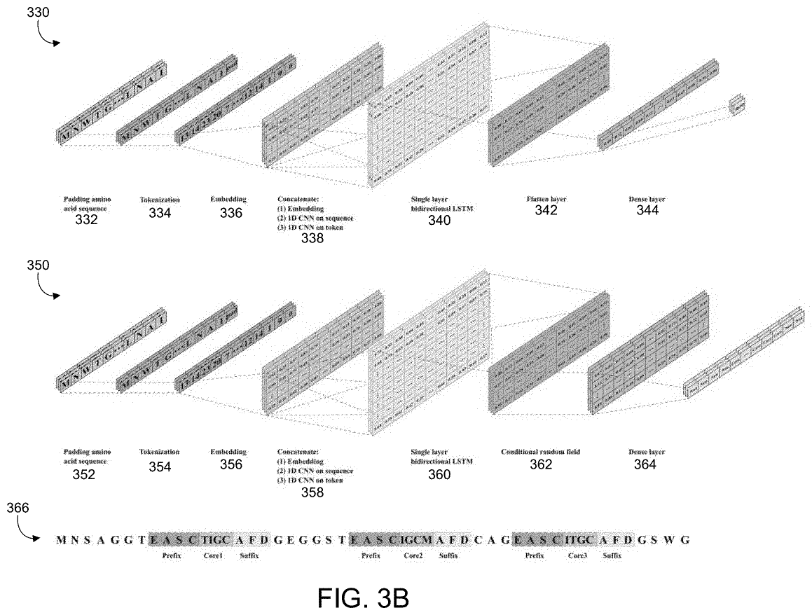

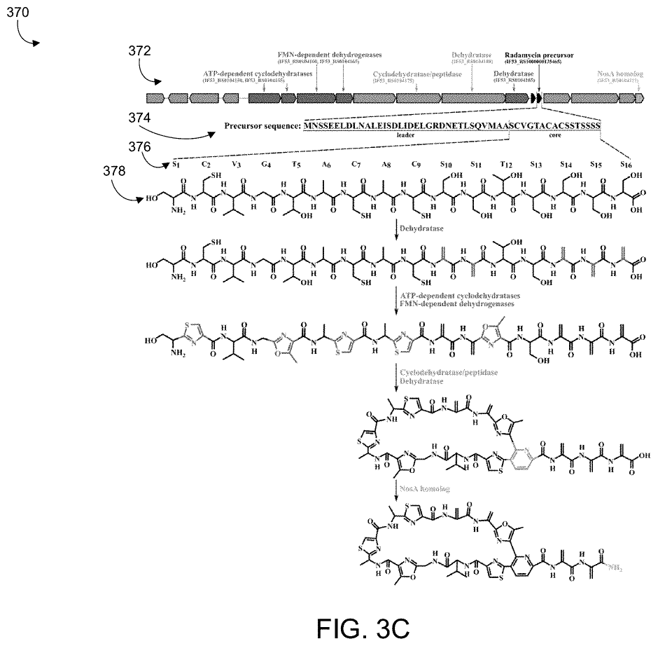

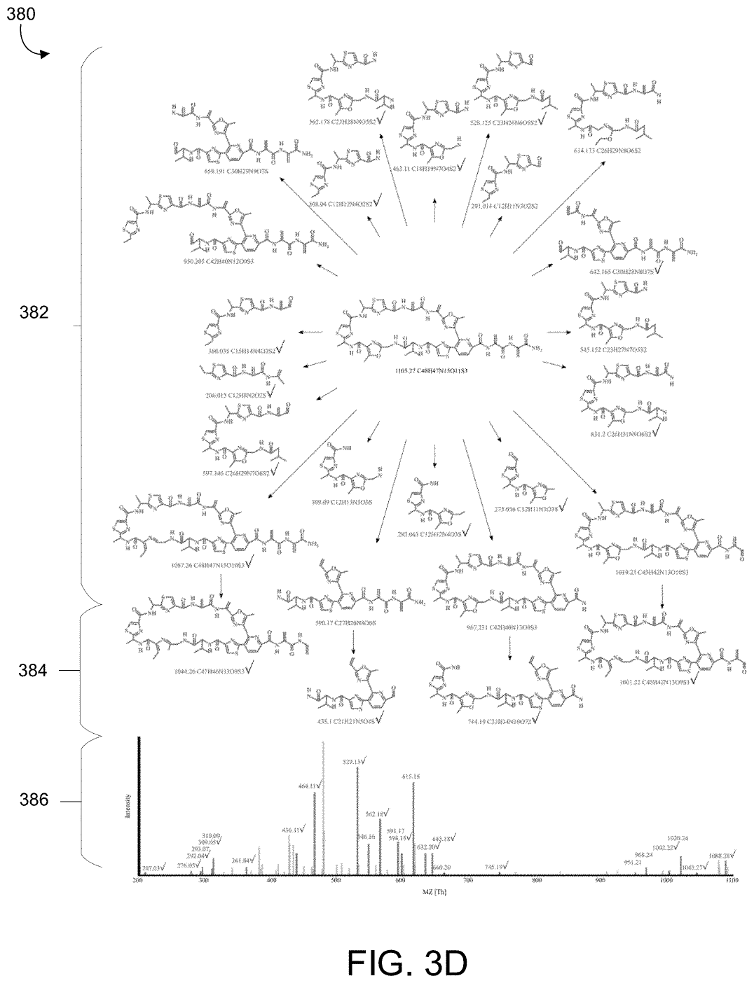

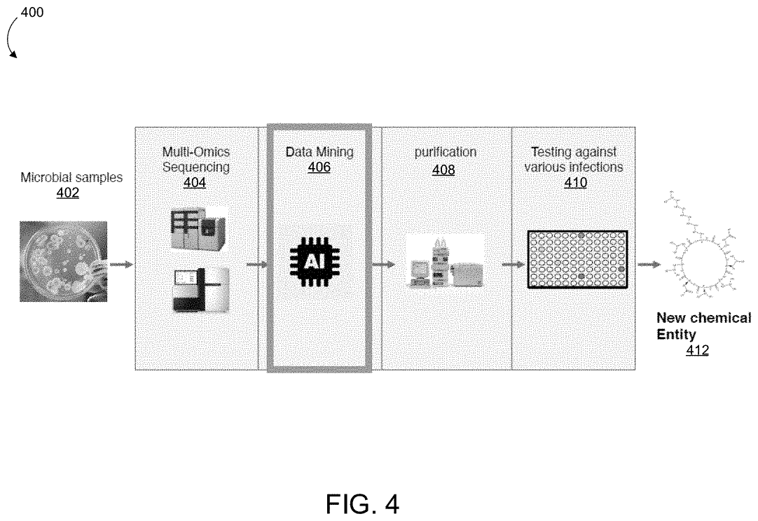

The data processing system 102, described in further detail below, is configured to receive the genomics data 106 and the metabolomics data 108 and generate associations between identified natural products of the natural products data 110 and the extracted spectra of the spectra data 112. More specifically, the data processing system 102 is configured to generate metabolite graphs and generate fragmentation graphs. These processes are described in greater detail in relation to FIGS. 3A-3B.

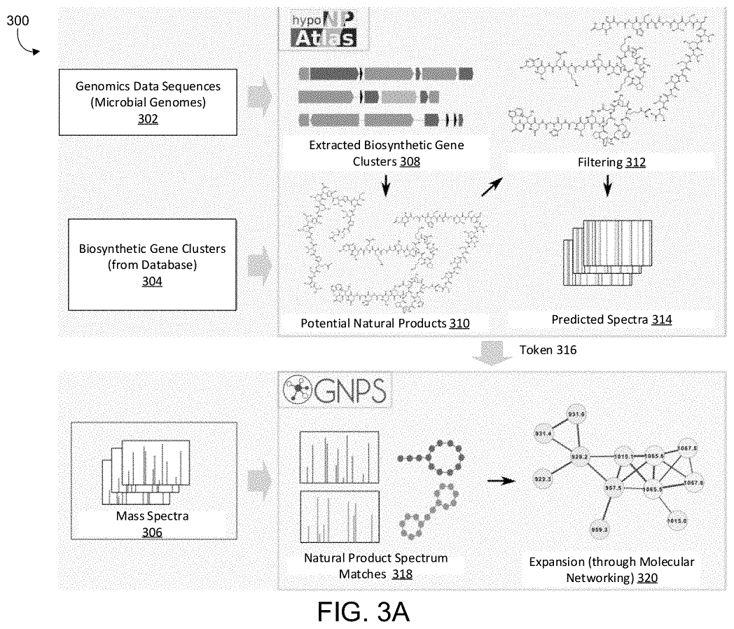

To generate the fragmentation graphs, in some embodiments the data processing system 102 uses a machine learning model configured to determine bridges and 2-cuts in the metabolite graphs that are more probable than other possible bridges or 2-cuts in the fragmentation graph. As described in relation to FIG. 2, a process 200 including machine learning models, such as a probabilistic model, is executed by the data processing system 102 for matching fragmentation graphs of the metabolomics data and mass spectra. The matches or pairs of fragments and mass spectra are scored by the data processing system 102. The scoring process is further described in relation to FIG. 1B.

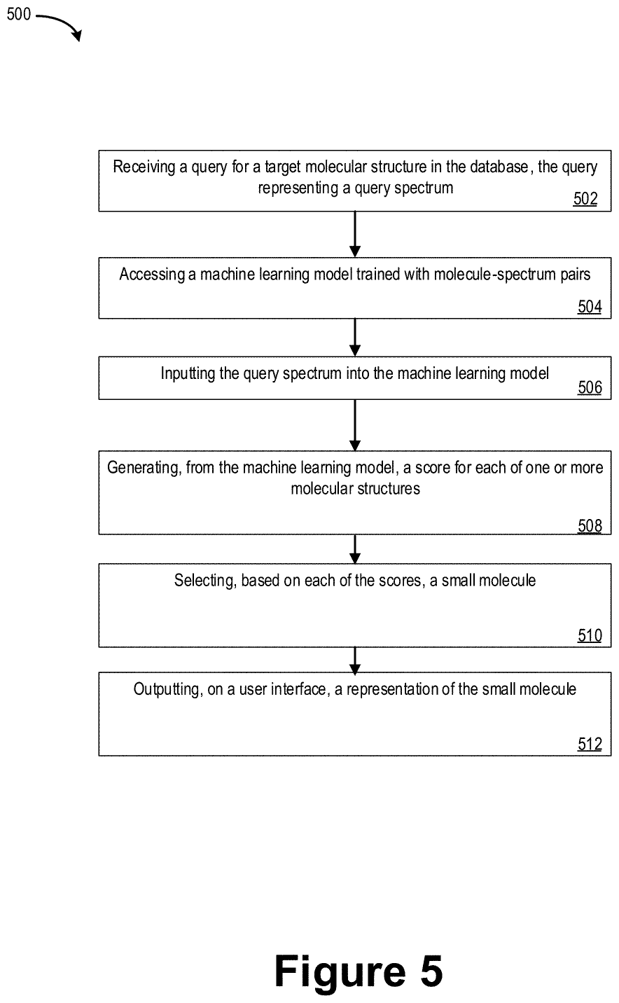

The data processing system 102 is configured to identify one or more natural products based on the scoring. In some implementations, the data processing system 102 outputs a ranked list of potential natural products. Once the natural product(s) of the sample are identified, the sample can be purified and tested against various infections to complete the process of drug discovery, described in relation to FIG. 4.

FIG. 1B shows an example process 150 performed by the data processing system 102 for identifying the structures of molecular compounds from mass spectrometry data using the system of FIG. 1A. Steps 152, 154, 156, 158, 160 and 161 show a training procedures for training the probabilistic model of the data processing system 102. Steps 162, 164, 166, 168, 170 and 172 are the scoring procedures based on the pre-trained probabilistic model. At step 152, a reference molecule R in the spectra is identified. At step 154, a reference spectrum of R is obtained. At step 158, the fragmentation graph of R is generated. A root node represents whole molecule, while the other nodes are fragments of it. The edges are annotated with the type of bonds where the fragmentation occurs. A fragment is annotated if it corresponds to a mass peak in the reference spectrum with a logrank of 1, 2, 3, or 4 respectively.

At step 156, an annotation of the reference spectrum of R is shown. A mass peak is annotated with a fragment if its mass is identical to the mass of the fragment plus charge, within a tolerance. As step 160, a table is generated that counts the number of fragments observed in training data with a specific bond type and an associated logrank. This table is called a count matrix. Because the number of peaks is small, the peaks have logranks of 1-4. The absent ones are shown with the lowest possible logrank=7. Logranks between 1-6 are allowed corresponding to peaks with ranks between 1-64. The null column corresponds to the experimental peaks which cannot be explained by the fragmentation graph. At step 161, the probabilistic model P(logrank|bondtype) is shown, which is determined by normalizing the count matrix.

At step 162, a molecule Q in a chemical structure database is obtained. At step 164, a query spectrum is obtained. At step 166, a fragmentation graph of Q is generated. At step 168, an annotation of the query spectrum with the fragmentation graph of Q is generated. At steps 170 and 172, a computation of the query spectrum match score is generated. This is the sum of scores of all the fragments in the annotated fragmentation graph. The P(Logrank=i|bondtype) is represented by P (i|bondtype).

Constructing Fragmentation Graphs of Small Molecules

The data processing system 102 constructs a metabolite graph for a small molecule structure and then generates a fragmentation graph from the metabolite graph. To simplify the modelling of small molecule fragmentation, mass spectrometry is assumed to break only N—C, O—C and C—C bonds.

To construct a metabolite graph, the data processing system 102 disconnects N—C, O—C and C—C bonds. The resulting connected components form the nodes of the metabolite graph. Edges in the metabolite graph correspond to bonds between the connected components.

The fragmentation graph of a molecule is a directed acyclic graph with a single source (the metabolite graph) where nodes are fragments of the molecule and directed edges between the nodes represent bridge or 2-cut fragmentations. To construct a fragmentation graph, the data processing system 102 searches for all the bridges and 2-cuts of the metabolite graph to obtain depth one fragments of the molecule (e.g., using Hoperoft and Tarjan's algorithm). Each fragment of the molecule is represented by a fixed length binary vector that indicates the presence or absence of metabolite graph nodes in the fragment. Depth-two fragments are formed by a bitwise AND operation between their parent fragments and depth one fragments. This generalizes to computing fragments at depth n>−1 by intersecting their parents (fragments at depth n−1) with fragments at depth 1. The final fragmentation graph is constructed by connecting the source node to all depth one fragment nodes and then iteratively connecting depth n−1 fragment nodes to the corresponding fragments at depth n.

Learning a Probabilistic Model to Match Mass Spectra and Small Molecules.

The data processing system 102 uses a naive scoring scheme which ignores that (i) fragmentation probability of different bonds are different, (ii) bridges have a higher chance of fragmentation than 2-cuts, (iii) peaks with higher intensity have higher chances of corresponding to a fragmentation than lower intensity peaks, (iv) matches are biased towards molecules, with large fragmentation graph size, and (v) matches are biased towards spectra with a large number of peaks. A probabilistic model is developed to overcome these issues for matching spectra with small molecules. Given a molecule-spectrum pair in the training data, a fragmentation graph of the molecule is constructed using the fragmentation graph construction algorithm previously described. Each fragment in the fragmentation graph is assigned a (bond type, logRank) label.

BondType represents the bond(s) disconnected in the parent fragment to produce the current fragment. BondType can either be one bond (bridge) or two bonds (2-cut). Bridges can be OC, NC, CC, while 2-cuts can be their pairwise combinations.

LogRank represents the intensity of the mass peak corresponding to the fragment. Intensity rank is an abundance measure of mass peaks in a spectrum. The better the intensity rank is (closer to rank 1), the more abundant the corresponding fragment. To reduce the number of parameters and avoid overfitting, peaks are grouped according to their log Rank instead of directly using intensity rank. A fragment is annotated with a logRank between 1-6 if there is a peak with a rank between 1-64 in the spectrum that is also within 0.01 Da of the mass of the fragment. If there is no such mass peak, the fragment is annotated with logRank=7.

In the annotated fragmentation graph, the logRank of each fragment depends only on its bondType and the logRank of its parent. The direct parent is considered. Considering grandparents of the fragments increases the number of parameters by an order of magnitude, resulting in overfitting. Additionally, a logRank of each mass peak is independent from the logRank of other peaks. This assumption simplifies the probabilistic model. Finally, the root node has logRank 0.

Scoring a Spectrum Against a Small Molecule.

Given a query tandem mass spectrum and a small molecule in a chemical structure database, if the spectrum precursor mass is within 0.02 Dalton of the mass of the small molecule and the user-specified adduct, these become a candidate pair for scoring. The data processing system 102 generates the fragmentation graph of the molecule and annotates the query spectrum against the fragmentation graph. Based on the described probabilistic model, a log-likelihood ratio model is used to score the spectrum against the small molecule:

Computing FDR.

Target-decoy analysis is used to estimate FDR. The data processing system 102 randomly shuffles the fragmentation graph of target molecules to create decoy fragmentation graphs. The data processing system 102 searches tandem mass spectra against the target and the decoy fragmentation graph databases respectively. At each score cutoff, there are target matches to the target database and N_{decoy} matches to the decoy database, and the ⋅FDR is estimated as N_{decoy}/N_{target}.

FIG. 2 shows an example process 200 for executing a trained machine learning model to identify the structures of molecular compounds from mass spectrometry data using machine learning models. The model is configured for learning a fragmentation pattern of small molecules in mass spectrometry based on a message-passing neural network (MPNN) framework for graph-structured data. The data processing system 102 is therefore an efficient database search tool for de-replication of small molecules based on corresponding associated mass spectra.

The process 200 is executed by the data processing system 102. As shown in the process 200, an input mass spectrum 202 is converted to a feature set 204 using either the dense or sparse representational method. In the machine learning model, the spectra is represented using the peak masses and their intensities. This spectral representation is then encoded into a continuous latent space via a neural network 206. The * denotes convolution. An input molecule 208 is converted into a molecular graph with node and edge labels 210. The data processing system 102 encodes the molecular graph into a continuous latent space by a message passing neural network 212. The data processing system 102 uses a concatenation of spectral 206 and molecular 212 embeddings as input an into a machine learning model. The machine learning model includes a sigmoid activation configured to generate outputs as scalar scores representative of a probability of the input spectrum and the chosen small molecule being a true match 214. The probability analysis is described in greater detail in relation to FIGS. 3A-3D.

A case study of the MPNN framework is now described which shows examples of improved efficacy and reduced processing time relative to existing models. In this particular example, unique spectra were selected that have valid molecular annotations and a [M+H]+ adduct from existing natural products libraries (e.g., in the Global Natural Product Social Molecular Networking library). The filtering performed by the data processing system 102 resulted in 4,437 molecule-spectrum pairs (corresponding to natural products data 110 and spectra data 112 of FIG. 1). These molecule spectrum pairs were used as positive-label training data. The training data were augmented with decoy molecules from available chemical databases including over 100,000,000 unique compounds. Spectra were paired with decoy molecules that had masses within 0.02 Da of a true molecule's mass, and the data processing system 102 assigned a negative label to those decoys. The machine learning models were training with 5-fold, leave-one-out cross-validation. For evaluation of all tested de-replication examples using the process 200, the data processing system 102 search the GNPS spectra against 76,446 unique molecules from a dictionary of natural products (DNP).

The process 200 was evaluated by searching the database of GNPS spectra and the DNP database. The data processing system 102, using the sparse spectral encoding and the modified GGNN molecule encoding with a hidden state size of 800, trained a dropout of 30% for 500 epochs, atop 1 accuracy of 48.8% was achieved in this example in 1.9 minutes.

Various processes for searching GNPS spectra against DNP are shown in Table 1, below. When running on just a single central processing unit (CPU), the process 200 is faster than the next fastest method.

TABLE 1
Total single-threaded runtime and peak
memory usage for each tested method when
searching 4,437 spectra from GNPS against
76,446 molecules from DNP.

| | Running | Peak |
|---|---|---|
| | time | Memory |
| Method | (d-h:m:s) | (gigabytes) |
| Process 200 (GPU) | 00:04:25 | 3.19 |
| Process 200 (CPU) | 00:13:40 | 0.46 |
| Process 150 | 00:29:04 | 0.15 |
| CFM-ID | 00:59:37 | 0.012 |
| Dereplicator+ | 04:28:10 | 19.98 |
| MAGMa+ | 2-20:34:37 | 0.51 |
| MetFrag | 12-08:57:24 | 4.01 |
| CSI:FingerID | 29-09:35:05 | 19.68 |

An example of a particular MPNN is now described. Let G=(V, E) be an undirected graph with nodes vᵢ ∈ V and edges e_{i,j} ∈ E. Let ne(i) be the set of nodes neighboring vᵢ. Let co(i) be the set of edges coincident to vᵢ. Molecular structures are represented by labeled graphs G=(V,E,L_{V},L_{E},l), where lᵢ ∈ L_{V} denotes the label for node i and l_{i,j} ∈ L_{E} denotes the edge label for e_{i,j}. Let [x]={1, 2, . . . , x}.

A message-passing neural network (MPNN) is defined by a message passing function Mₜ, an update function Uₜ, and a readout function R. A hidden state t, h^{t} ∈ R^{H}, is assigned to each node vᵢ in the input graph with equations 1 and 2.

$$\begin{matrix}{m}_{i}^{t} = {\sum }_{j ∈ ne ( i )} {M}_{t - 1} ( {h}_{i}^{t - 1} , {h}_{j}^{t - 1} , {ℓ}_{i , j} ) & ( 1 ) \\ {h}_{i}^{t} = {U}_{t - 1} ( {h}_{i}^{t - 1} , {m}_{i}^{t} ) & ( 2 )\end{matrix}$$

for time steps t [T]. Typically, T is a user-defined hyperparameter. In the first iteration h⁰ is set Wᵢfᵢ where Wᵢ R^{H×|LV|} is a learned weight matrix that converts the graph label dimension to the MPNN hidden state dimension. After T message-passing steps a graph-level hidden state is computed via readout function R. Note that in order for the graph-level hidden state to be robust to isomorphism R must be an order-invariant function, such as an element-wise sum or mean. The gated graph neural network (GGNN) is a special case of the MPNN, defined by Equations 3-5.

$$\begin{matrix}{M}_{t} = {f}_{{ℓ}_{i , j}} ( {h}_{j}^{i} ) & ( 3 ) \\ {U}_{t} = GRU ( {h}_{i}^{t - 1} , {m}_{i}^{t} ) & ( 4 ) \\ R = tanh ( {\sum }_{{v}_{i} ∈ V} σ ( α ( {h}_{i}^{T} , {ℓ}_{i} ) ) ⊙ tanh ( β ( {h}_{i}^{T} , {ℓ}_{i} ) ) ) & ( 5 )\end{matrix}$$

where f [symbol] _{i,j} is a multi-layer perceptron (MLP) defined for each possible categorical edge label, GRU is a gated recurrent unit, σ is the sigmoid activation function, and α and [symbol] are multi-layer perceptrons (MLPs).

The MPNN executed by process 200 generates a scoring function that accepts a molecule (e.g., of metabolomics data 108) and a mass spectrum (e.g., of database 104) as input and outputs a score that correlates with the likelihood that the mass spectrum is generated from the molecule. To score a (molecule, mass spectrum) pair, the data processing system 102 separately embeds the molecular graph and mass spectrum into continuous latent spaces with a molecule encoder network and a spectrum encoder network, respectively.

The data processing system 102 process 200 represents molecules as undirected labeled graphs. The data processing system 102 uses a MPNN for the molecule encoding network. In an example, Node and edge level features from Table 2 are used. For each input molecule we employ a modified GGNN that allows for arbitrary continuous edge-labels defined by equations 6-9.

$$\begin{matrix}{h}_{i}^{0} = [ {ℓ}_{i} , 0 ] & ( 6 ) \\ {h}_{i , j} = {W}_{e} {ℓ}_{i , j} & ( 7 ) \\ {m}_{i}^{t} = {\sum }_{j ∈ ne ( i )} ReLU ( {W}_{h} [ {h}_{j}^{t - 1} , {h}_{i , j} ] ) & ( 8 ) \\ {h}_{i}^{t} = GRU ( {h}_{i}^{t - 1} , {m}_{i}^{t} ) & ( 9 )\end{matrix}$$

where [⋅,⋅] indicates concatenation, Wₑ and Wₕ are learned weight matrices, and ReLU is the rectified linear unit activation function. R is a readout function.

TABLE 2
Feature Set for Structures.

| Feature Type | Feature | Size |
|---|---|---|
| Atom | 1-hot Atomic Number | 100 |
| | 1-hot Number of Bonds | 6 |
| | 1-hot Formal Charge | 5 |
| | 1-hot unspecified, tetrahedral CW/CCW, | 4 |
| | or other chirality | |
| | 1-hot Number of Hydrogens | 5 |
| | 1-hot sp, sp2, sp3, sp3d, sp3d2 Hybridization | 5 |
| | Binary Aromaticity | 1 |
| | Atomic Mass Divided by 100 | 1 |
| Bond | 1-hot single, double, triple, aromatic Bond Type | 4 |
| | Binary Conjugated | 1 |
| | Binary In Ring | 1 |
| | 1-hot none, any, E, Z, cis, trans, | 7 |
| | unknown Stereochemistry | |

An example spectrum encoding network of the data processing system 102 is now described. Two example processes are described for representing and encoding mass spectra. First, in the sparse representation, the data processing system 102 generates features from each mass spectrum as a sequence of m/z, wherein intensity tuples sorted by descending intensity. Intensities are normalized to lie in [0, 1]. The data processing system 102 filters the spectra (based on the process previously described) to retain a top P most intense peaks (e.g., P=20). In some implementations, the spectrum encoder can be executed by a single GRU cell applied sequentially to the concatenated m/z and normalized intensity. The hidden state after reading the last input is used as the latent representation of the spectrum.

An alternative strategy is the dense representation. The data processing system 102 discretizes the m/z axis by combining peaks in non-overlapping windows of size δ by summing their intensities. The intensities are normalized as described previously. The data processing system 102 sets an m/z upper bound of 5000 Da to ensure all mass spectra have a same number of features. In some implementations, the spectrum encoder is a one-dimensional convolutional neural network.

The scoring network. The scoring network is a MLP where the input is the concatenation of the latent representations computed by the molecule encoding and the spectrum encoding network. This network has ReLU activations at each hidden layer and a sigmoid activation for the output layer.

The data processing system concatenates the two latent representations and feeds the result into a scoring network that outputs a scalar indicating the molecule-spectrum match (MSM) score of the input pair, as previously described. The whole architecture is trained end-to-end with a binary cross-entropy loss, where training labels indicate if the spectrum is truly generated from the molecule.

FIG. 3A shows an example process 300 for training the machine learning model of FIG. 2 to identify the structures of molecular compounds from mass spectrometry data. The process 300 can be executed by data processing system 102 of FIG. 1A and is an example of the process described in FIG. 1B. In this example, the compounds include ribosomally synthesized and post-translationally modified peptides (RiPPs), or ribosomal natural products. For this example, the process 300 is configured for novel RiPP discovery. At step 308, the data processing system 102 extracts biosynthetic gene clusters (BGCs) from available metabolomics data 302, 304. A BGC includes a locally clustered group of two or more genes that together encode a biosynthetic pathway for the production of a secondary metabolite from microbial examples. At step 310, the data processing system 102 builds associations between structures and spectra by predicting hypothetical molecule structures from the BGCs. At step 312, the potential natural products are filtered to specific taxonomies or gene clusters based on the taxa- or meta-genomic data available from the samples of interest. At step 314, the data processing system 102 predicts mass spectrometry fragmentation of the hypothetical natural products, along with known RiPPs the data 304.

The data processing system 102 generates 316 a token representing the predicted spectra and natural products pairs. The data processing system 102 collects mass spectra data 306 for the environmental samples or the microbial isolates. At step 318, the data processing system 102 searches the mass spectra 306 against the predicted spectra data from step 314 of hypothetical molecules. The data processing system 102 outputs high-scoring RiPP-spectrum matches. Here, high scoring matches include matches above a threshold correlation score or a list of the most likely candidates, regardless of score. The identifications are further expanded at step 320 by the data processing system 102 through propagation in the molecular network. In an example, steps 308, 310, 312, and 314 performed once to prepare the machine learning models. The data processing system 102 stores the relationships stored in a repository (such as database 104). Steps 306, 318, and 320 are repeated for every new mass spectral dataset.

Overall, the process of generating hypothetical RiPPs from genomes includes four steps. The data processing system 102 extracts BGCs from the input genome by searching RiPP related genes. The data processing system 102 then extracts all ORFs from each BGC and identifies the RiPP precursor ORFs. The data processing system 102 then identifies the potential cleavage site within the ORFs and produces cores. The data processing system 102 generates hypothetical chemical structures of the RiPP from the core and the enzymes in the BGC. More specifically, the data processing system 102 identifies RiPP BGCs based on the biosynthetic enzymes from a microbial genome sequence. The data processing system 102 identifies RiPP precursor ORFs from a BGC. The data processing system 102 identifies RiPP core peptides from an ORF. The data processing system 102 generates a combinatorial list of feasible mature RiPPs for each core peptide based on the tailoring enzymes present in the BGC. These steps are now described in greater detail.

The data processing system 102 extracts RiPP BGCs in the following steps. The data processing system 102 translates the genome sequence is translated in six frames. The data processing system 102 identifies RiPP related proteins through a search. The data processing system 102 defines contigs by extracting genome sequence from the middle of the protein to 10,000 bp upstream and downstream. Contigs include a series of overlapping DNA sequences used to make a physical map that reconstructs the original DNA sequence of a chromosome or a region of a chromosome. The data processing system 102 identifies BGCs after merging overlapping contigs.

The data processing system 102 identifies RiPP precursor ORFs in the following steps. The data processing system 102 translates the DNA sequence of the BGC in six frames. The data processing system 102 identifies RiPP biosynthetic enzymes using a search. The data processing system 102 extracts ORFs in the vicinity of the biosynthetic enzymes (default 10,000 base pairs). The data processing system 102 identifies candidate RiPP precursors are identified using ORF prediction tools. The data processing system 102 filters this list of candidate ORFs using a deep neural network, shown in FIG. 3B.

The data processing system 102 predicts RiPP core peptides from their ORFs in the following steps. The data processing system 102 identifies top k N-terminal and C-terminal cleavage sites from each ORF using a deep neural network (k is a user-defined threshold). The data processing system 102 generates a combinatorial list of putative core sequences (up to k2 cases). When the precursor contains repetitive patterns (e.g. cyanobactins and plant RiPPs), the data processing system 102 uses a repeat-specific core finding strategy to identify core sequences from repeated prefix and suffix patterns

FIG. 3B shows examples of a machine learning models 330, 350, for identifying natural products from genomic sequences 366. More specifically, FIG. 3B shows deep neural networks (DNNs) used to identify candidate ORFs from genomic sequences. The model 330 includes a padding process 332, a tokenization 334, an embedding layer 336, two 1D convolutional neural networks (CNNs) 338, a single layer bidirectional long short-term memory (LSTM) network 340, a flatten layer 342, and a dense layer 344. This model 330 takes an ORF peptide sequence as input, and classifies the peptide as RiPP or non-RiPP ORF.

An example of model 330 is now described. In an example, all input sequences are padded to a length of 200 amino acids. Each amino acid, including the padding symbol, is tokenized and embedded into a vector of size 100. The model 330 includes two 1D CNNs. One CNN convolves the input sequence in terms of its topology, the other CNN convolves the tokenized vectors. The convolution on the sequence helps the model to detect the RiPP features on amino acids; the convolution on the token vector summarizes the embedded character information in high dimensional space. The outputs from the two CNNs and the input embeddings are concatenated and fed into a single layer bidirectional LSTM. The LSTM learns and summarizes the sequential features from the amino acid chain. The output of the LSTM is flattened and converted to a binary output with a flattened layer and a dense layer. The prediction loss is calculated by cross entropy loss during the training of the model. The learning rate begins with 1e-3 and decreases 10% every 40 epochs.

The model 350 includes a padding process 352, a tokenization 354, an embedding layer 356, two 1D CNNs 358, a single layer bidirectional LSTM 360, a conditional random field layer 362 and a dense layer 364. This model 350 takes an ORF as input, and identifies k N-terminal and k C-terminal cleavage sites, where k is a user-defined value. Sequence 366 represents an alternative core finder used to search repeated (at least twice) prefix-suffix patterns and identify the core sequence in the patterns.

An example of model 350 is now described. A discriminatory deep learning model predicts core and non-core frames of sequences given the amino acid sequence of an ORF as the continuous input. All input sequences are padded to a length of 200 amino acids. Each amino acid is tokenized and embedded into a vector of size 25. The model 350 includes two 1D CNNs. One CNN convolves the input sequence in terms of its topology, and the other CNN convolves the tokenized vectors. The convolution on the sequence helps the model to detect amino acids surrounding the enzymatic cleavage site. The convolution on the token vector summarizes the embedded character information in high dimensional space. The outputs from the two CNNs, and the input embeddings are concatenated and fed into a single layer bidirectional LSTM. The LSTM learns the translation from the amino acid chain to core and non-core frames of sequences. The prediction loss is calculated by a conditional random forest layer, which calculates the negative log-likelihood during the training of the model, and performs the Viterbi algorithm to optimize labels during prediction.

FIG. 3C shows an example process 370 for determining a molecular structure based on genomic data. At step 372, the data processing system 102 identifies BGCs through their modification enzymes. For example, dehydration enzymes are frequently found in some classes of RiPPs. At step 374, short ORFs within 10,000 bp of these enzymes are detected by the data processing system 102 as candidate structural ORFs. At step 376, fragments of the structural ORFs with lengths between 5 to 30 amino acids are extracted by the data processing system 102 as candidate precursor peptides. The data processing system 102, depending on the tailoring enzymes in the BGC, optionally applies corresponding modifications to the precursor peptides to form hypothetical molecules

FIG. 3D shows an example process 380 for mapping structures of molecules to spectra data. More specifically, FIG. 3D shows an annotation of radamycin mass spectra from GNPS actinomycete dataset MSV000078937 using a dereplicator+ model. The fragments at depth 1 are shown in bracket 382, while the fragments at depth 2 are shown in bracket 384. Fragments and peaks that are annotated by dereplicator+ but not by the string scoring method are marked with checkmarks. These can either correspond to depth two fragments or fragments resulting from non-amide bond breakage. Associated spectra 386 are shown below. By switching from a string-based model to this graph based model, the score of correct radamycin match increases from 9 to 25, and the p-value drops from 3×10⁻¹⁷ to 3×10⁻⁴⁶.

The data processing system 102 derives a complete chemical structure graph of RiPPs from their BGC (rather than a precursor peptide along with modification masses). To enable this, the data processing system 102, based on precursor peptides, models tailoring enzymes as graph-modifiers. Each enzyme searches a particular chemical motif in the molecular graph of a RiPP, and whenever it finds the motif, it optionally applies the corresponding tailoring modification.

The data processing system 102 predicts hypothetical molecules by applying modifications corresponding to tailoring enzymes present in the BGC. The data processing system 102 does this by extracting all the information from the known RiPP tailoring enzymes and their corresponding modifications. Given a core sequence and a list of tailoring enzymes the data processing system 102 predicts all the hypothetical RiPP structures using the following steps. First, the chemical structure of the core sequence is represented as a graph, where each vertex is an atom with an index number and the type of atom, and each edge is a bond between two indexed atoms and the type of chemical bonds. Next, for each modification, the location of motifs are collected by subgraph isomorphism. Finally, the putative combinations of modifications are calculated and applied to the core sequence.

Estimating the Statistical Significance of Metabolomics Annotations

In case of mass spectrometry base proteomics, modern database search strategies recruit statistical methods to evaluate the matches between peptides and spectra. Currently, the dominant technique for statistical evaluation of a set of PSMs is to estimate false discovery rate (FDR) using the Target Decoy Approach (TDA). In this approach, a database of fake (decoy) peptides are searched against mass spectra in addition to correct (target) database. Then the ratio of high score matches in the decoy database to that of target database is used as an estimate of FDR. Decoy peptides are usually generated by shuffling target peptides. Methods below are discussed to generalize this method to other natural products with more diverse structures.

In proteomics, identified PSMs are statistically evaluated through the computation of p-values. The p-value of a PSM is defined as the fraction of random peptides with a score equal to or exceeding the target PSM. To compute p-value, the data processing system 102 estimates the distribution of the score of random peptides against the spectrum.

Natural product small molecules have diverse graph structures, such as linear, cyclic, branch-cyclic, and multiple ring structures. The process now described is configured for statistical validation of metabolomics identifications by converting match scores to p-values. This process overcomes existing technical hurdles. Current methods (such as Markov Chain Monte Carlo (MCMC)) for estimating p-values do not generalize to other types of natural products since it is not clear how to generate random small molecule structures, and how to randomly modify the structure of small molecules. To overcome this, the data processing system 102 is configured to use constrained graph variational autoencoders (CGVAE) for random generation and mutations of small molecules.

Matching Score and P-Values

A scoring function score(M, S) for a match between molecule M and spectrum S. Let M and S denote the set of all possible molecules and spectra. Molecule-based p-value for a pair of molecule and spectrum (M, S) is defined as:

[symbol]

(Score(m, S)≥t) (10)

where m is generated from a random uniform distributed on set and =Score (M, S). The probability p defined above depends on the choice of set M. Similarly, spectrum-based p-value is defined as:

[symbol]

(Score(M, s)≥t) (11)

where s is generated from a random uniform distributed on set. Molecule-based p-values help in reducing bias toward molecular features, while spectrum-based p-values reduce bias toward spectral features. The data processing system 102 uses constrained graph variational autoencoders (CGVAE) for generating random molecules, and also generates random spectra.

Monte Carlo Approach

Monte Carlo simulation is a robust technique for simulating events and estimating their probabilities. Consider the set

[symbol]

*={m ∈ [symbol] : Score(m, S)≥t} (12)

S*={s ∈ S: Score(M, s)≥t} (13)

Define 1M* as an indicator function of the set M* such that 1M*(m) equals 1 if m ∈ M* (equivalently, Score(m)≥t) and 0 otherwise. Let m1, . . . , m_{N} be N independent and identically distributed (iid) random variables sampled from a uniform distribution f on set M. The p-value p defined in equation (10) can be written as an expectation:

p= [symbol] (m)r(m)dm= [symbol] [ [symbol] ] (14)

where r is the sampling distribution over M. According to the strong law of large numbers, since m1, . . . , mN are iid samples, the probability p converges almost surely (i.e., with probability 1) to the following:

$${\hat{p}}_{MC} = \frac{1}{N} {\sum }_{i = 1}^{N} {{ℳ}^{o}}_{} ( {m}_{i} ) \qquad ( 15 )$$

\hat{p}_{MC} is an unbiased and consistent estimator of p-value p. Variance of \hat{p}_{MC} equals to p(1−p), which tends to 0 as N. Therefore Monte Carlo sampling is used to compute the probability p.

Importance Sampling

Importance sampling is a general Monte-Carlo approach for reducing the variance of expectations quantities. Importance sampling generates the events of interest more often by sampling from a different distribution and correcting for the bias afterward, which results in a more accurate estimate with a lower number of samples. Formally, assume m₁, . . . , m_{N} are sampled from a distribution with density p. Then for a probability distribution q(m), using equation (16):

$$p = {\int }_{ℳ} {{ℳ}^{*}}_{} ( m ) r ( m ) d m = {\int }_{ℳ} \frac{{{ℳ}^{o}}_{} ( m ) r ( m )}{q ( m )} q ( m ) d m = {𝔼}_{q} [ \frac{{{ℳ}^{o}}_{} r}{q} ] \qquad ( 16 )$$

The importance sampling estimator of p is defined as:

$${\hat{p}}_{IS} = \frac{1}{N} {\sum }_{i = 1}^{N} \frac{r ( {v}_{i} )}{q ( {v}_{i} )} {𝒮}_{} ( {v}_{i} ) = \frac{1}{N} {\sum }_{i = 1}^{N} w ( {v}_{i} ) {𝒮}_{} ( {v}_{i} ) \qquad ( 17 )$$

where v1, . . . , vN are iid samples from q(v), called the importance sampling density, and w (vi)=p(vi) is the importance weight of sample vi.

Markov Chain Monte Carlo

The importance sampling density q has is selected in a way that relative error is minimized. The optimal density is:

$${q}^{*} ( m ) \frac{{{ℳ}^{*}}_{} ( m ) r ( m )}{p ɛ} \qquad ( 18 )$$

In this case, all generated samples fall in Uᵢ ∈ M*, so their importance weights are w (mi)=p, and the IS estimate \hat{p}_{IS}=p. In this case, just one sample (N=1) is enough to find the probability p exactly. To estimate the optimal importance sampling density, the data processing system 102 uses an iterative strategy. In the first iteration, a uniform distribution q is used, and in each iteration, the score probability distribution P(Score(m, S)=t) is estimated, and the weight and density are updated as:

$$\begin{matrix}w ( t ) = \frac{1}{ℙ ( Score ( m , S ) = t )} & ( 19 ) \\ q ( t ) = r ( t ) ℙ ( Score ( m , S ) = t ) & ( 20 )\end{matrix}$$

Here, we assume density q and weight w are uniform for each score. Usually this task is approached by using the Metropolis-Hastings algorithm that allows for the usage of unnormalized densities. The data processing system 102 is configured to generate a Markov Chain having Q as an equilibrium distribution and obtain the desired sequence {vₙ} of random variables via sampling from this distribution. The ergodicity of the Markov chain ensures that as N approaches infinity, \hat{p}_{IS} still converges to the target probability p.

Consider a transition kernel γ(x|y) that defines a jump in M. The Metropolis Hastings algorithm realizes the desired sampling strategy as follows. Starting from a random initial point m₀, in each step a new point m* is samples from γ(x|y). If w(m*)>w(mᵢ), the transition is accepted. Otherwise the transition is accepted with probability w(m*)/w(mᵢ), and rejected otherwise. If the transition is accepted, mᵢ is set to m*, otherwise mᵢ is set to mi−1.

Algorithm 1: Metropolis-Hastings algorithm

Input: Transition kernel γ(x|y), current state of Markov Chain \vec{v}ᵢ
Output: Next state of Markov-Chain \vec{v}ᵢ₊₁
Sample random variable \vec{v} from conditional distribution γ (· | \vec{v}ᵢ)
Sample uniform random variable r on the interval [0; 1]
$Calculate the acceptance ratio α = min ( \frac{q ( {{v}^{->}}_{i} ) γ ( {v}^{->} \vert {{v}^{->}}_{i} )}{q ( {v}^{->} ) γ ( {{v}^{->}}_{i} \vert {v}^{->} )} , 1 )$
if r < α then
└ \vec{v}ᵢ₊₁ ← \vec{v}
else
└ \vec{v}ᵢ₊₁ ← \vec{v}ᵢ

where the sampling from proposal density γ(⋅|\vec{v}ᵢ) is performed as follows:

Algorithm 2: Si [symbol] ulation from conditional density γ (· | \vec{ν}ᵢ)

Input: Current state of Markov Chain \vec{ν} = (ν1, . . . , ν_{|V(G)|}) ∈

[symbol]

Output: Proposed state \vec{ν}
Sample index i uniformly on {1, . . . , |E(G)|}
Consider eᵢ ∈ E(G) where (v₁, v₂) are starting and ending
vertices
Sample δ uniformly on [−m (v₁); m (v₂)]
set \vec{ν}ᵥ_{₁} based on δ (Details need to be added here)

[symbol]

indicates data missing or illegible when filed

Gathering all the parts of the proposed method together we end with the following algorithm to compute \hat{p}_{IS}.

Algorithm 3: Importance Sampling estimator for p

Input: A molecule MP and spectrum S
Output: An estimate p [symbol] _{S} of statistical significance p and confidence
interval C_{N}
Construct a random molecule m based on CGVAE model
let Score(m) = Score(S, m)
Determine the set of weights w(m) using Wang-Landan algorithm
(Details need to be added here)
while Stopping criterion is not satisfied do
| Simulate next state \vec{ν}_{N} using Metropolis-Hastings algorithm (see
| algorithm 1)
|_ update the stopping criterion
Calculate \hat{p} [symbol] _{S} based on Equation 9

[symbol]

indicates data missing or illegible when filed

There are two ways for computing the statistical significance of a molecule spectrum match. A first method fixes the molecule and computes the ratio of random spectra getting a score higher than the target match (spectrum-based p-value). A second method fixes the spectrum and computes the ratio of random molecules getting a score higher than the target match (molecule-based p-value). In case of spectrum-based p-values, the data processing system 102 generates (and modifies) random spectra based on the statistics of peaks/intensities learned from the GNPS library. In the case of molecule-based p-values, the data processing system 102 uses constrained graph variational autoencoders (CGVAE) for random generation and modification of molecular structures.

Generating Random Spectra

The data processing system 102 determines a statistical significance for generated molecule structures by producing a target-decoy database of random MS/MS spectra for small molecules. For this part the set M includes of generated spectra in order to calculate equation (10) for each pair of (molecule, spectrum). Two strategies are used to create the decoy MS/MS database.

A first method to create the decoy MS/MS database is the naïve method. For the naive decoy spectral library, all possible fragment ions from the reference library of spectra are used. The data processing system 102 randomly adds these ions to the decoy spectral library until each decoy spectrum reaches the desired number of fragment ions that mimics the corresponding library spectrum. This method is presented as a baseline evaluation of the other, more intricate method.

The spectrum-based method is similar to the naive method, as the data processing system 102 generates a decoy spectral library through choosing fragment ions that co-appear in the spectra from the target spectral library. In this spectrum-based approach, the data processing system 102 starts with an empty set of fragment ion candidates. First, the data processing system 102 randomly selects the precursor fragment ion of the target spectrum. For each fragment ion added to the decoy spectrum, the data processing system 102 chooses all spectra from the target spectral library which contain this fragment ion, within a threshold (=5 p.p.m). From these spectra, the data processing system 102 uniformly draws (all fragment ions have the same probability to be drawn) five fragment ions that are added to the fragment ion candidate set. The data processing system 102 draws a fragment ion from the fragment ion candidate set and add it to the decoy spectrum, then proceed as described above until we reach the desired number of fragment ions that mimics the corresponding library spectrum. The two-step process of first drawing candidates, then drawing the actual decoy spectrum is introduced to better mimic fragmentation cascades and dependencies between fragments. Furthermore, it prevents that fragment-rich spectra dominate the process.

Generating Random Molecules

A CGVAE model is used by the data processing system 102 to generate random molecules. The process is seeded with N vectors that together form a latent specification for the graph to be generated (N is an upper bound on the number of nodes in the final graph). The model uses a variational autoencoder to design molecules that are close to the real molecules in the nature.

FIG. 4 shows an example process 400 for identifying a natural product drug from microbial samples. Process 400 shows natural product identification in the context of drug discovery. For example, microbial samples are collected (402). To identify natural products from these microbial samples, the samples are sequenced with multi-omics (404) processes. These processes generate genomic data including sequencing information and/or mass spectra of the samples. The sequences and mass spectra of the microbial samples are input into the data processing system 102. The data processing system 102 at step 406 is configured to determine the structures of the small molecules represented in the mass spectra based on the processes described above. Candidate structures can be purified (408) and tested (410) against various infections. A resulting drug can be determined as an output (412).

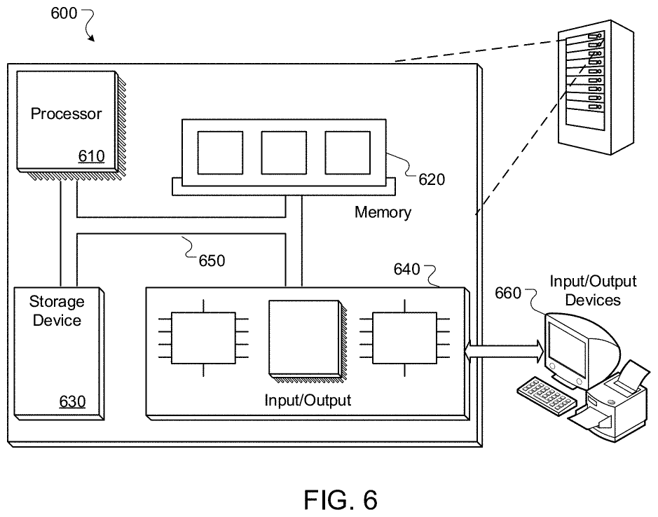

FIG. 5 shows a flow diagram representing a process 500 for identifying the structures of molecular compounds from mass spectrometry data. The process 500 can be executed by a computing system, such as the computing system described in relation to FIG. 6. The process can be executed by the data processing system 102 of FIG. 1. The process 500 is for searching a database to identify structures of molecular compounds from mass spectrometry data. The process 500 includes receiving (502) a query for a target molecular structure in the database, the query representing a query spectrum. In some implementations, the query spectrum is a spectrum generated from material in an environment for which the small molecular structures are unknown. The process 500 includes accessing (504) a machine learning model trained with molecule-spectrum pairs. A molecule-spectrum pair of the molecule-spectrum pairs includes structure data representing two-dimensional molecular structures of small molecules. The structure data is associated with spectrum data representing a mass spectrum that is generated from the small molecules represented in the structure data, as described previously. The process 500 includes inputting (506) the query spectrum into the machine learning model. The process 500 includes generating (508), from the machine learning model, a score for each of one or more molecular structures. Each score representing a probability that a molecular structure corresponds to the query spectrum. The process 500 includes selecting (510), based on each of the scores, a small molecule. The process 500 includes outputting (512), on a user interface, a representation of the small molecule.

In some implementations, the machine learning model enables a reduction in computing memory and a decrease in latency of returning the representation of the small molecule in response to receiving the query spectrum, the reduction being relative to a rule-based model using at least one of bond type data, hydrogen rearrangement data, and dissociation energy data for searching the database.

In some implementations, the process 500 includes training the machine learning model with the molecule-spectrum pairs. Training the machine learning model includes generating, for a given small molecule of the structure data, a fragmentation graph. The fragmentation graph representing one or more fragments of the small molecule. The one or more fragments correspond to mass spectrum peaks of the small molecule. The process 500 includes assigning each of the one or more fragments of the fragmentation graph, a bond type and a log rank. In some implementations, the bond type represents chemical bonds that are disconnected in a parent fragment to produce a fragment. In some implementations, the log rank represents an intensity of a mass peak corresponding to the fragment.

In some implementations, the fragmentation graph is generated for generating the machine learning model. Generating the fragmentation graph includes cutting, for the small molecule, N—C, O—C, and C—C bonds of the small molecule to generate sub-fragments to form a metabolite graph. Generating the fragmentation graph includes generating depth one fragments by searching the metabolite graph for each cut bond. Generating the fragmentation graph includes determining intersections between each depth one fragment and a corresponding parent fragment. Generating the fragmentation graph includes forming depth two fragments based on the intersection. Generating the fragmentation graph includes connecting the metabolite graph to each depth one node and each depth two node to a corresponding depth one node.

FIG. 6 shows an example computer system 600 that includes a processor 600, a memory 620, a storage device 630 and an input/output device 640. Each of the components 600, 620, 630 and 640 can be interconnected, for example, by a system bus 650. The processor 6100 is capable of processing instructions for execution within the system 600. In some implementations, the processor 600 is a single-threaded processor, a multi-threaded processor, or another type of processor. The processor 600 is capable of processing instructions stored in the memory 620 or on the storage device 630. The memory 620 and the storage device 630 can store information within the system 600.

The input/output device 640 provides input/output operations for the system 600. In some implementations, the input/output device 640 can include one or more of a network interface device, e.g., an Ethernet card, a serial communication device, e.g., an RS-232 port, and/or a wireless interface device, e.g., an 802.11 card, a 3G wireless modem, a 4G wireless modem, a 5G wireless modem, etc. In some implementations, the input/output device can include driver devices configured to receive input data and send output data to other input/output devices, e.g., keyboard, printer and display devices 660. In some implementations, mobile computing devices, mobile communication devices, and other devices can be used.

While this specification includes many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Moreover, the separation or integration of various system modules and components in the previously described implementations should not be understood as requiring such separation or integration in all implementations, and it should be understood that the described program components and systems can generally be integrated together in a single software product or packaged into multiple software products.

Accordingly, the previously described example implementations do not define or constrain the present disclosure. Other changes, substitutions, and alterations are also possible without departing from the scope of the present disclosure.

What is claimed is:

1. A computer-implemented method for searching a database to identify structures of molecular compounds from mass spectrometry data, the computer-implemented method comprising:
receiving a query for a target molecular structure in the database, the query representing a query spectrum;
accessing a machine learning model trained with molecule-spectrum pairs, wherein a molecule-spectrum pair of the molecule-spectrum pairs comprises structure data representing two-dimensional molecular structures of small molecules, the structure data being associated with spectrum data representing a mass spectrum that is generated from the small molecules represented in the structure data;
inputting the query spectrum into the machine learning model;
generating, from the machine learning model, a score for each of one or more molecular structures, each score representing a probability that a molecular structure corresponds to the query spectrum;
selecting, based on each of the scores, a small molecule; and
outputting, on a user interface, a representation of the small molecule.

2. The computer-implemented method of claim 1, wherein the machine learning model enables a reduction in computing memory and a decrease in latency of returning the representation of the small molecule in response to receiving the query spectrum, the reduction being relative to a rule-based model using at least one of bond type data, hydrogen rearrangement data, and dissociation energy data for searching the database.

3. The computer-implemented method of claim 1, further comprising training the machine learning model by performing operations comprising:
generating, for a given small molecule of the structure data, a fragmentation graph, the fragmentation graph representing one or more fragments of the small molecule, the one or more fragments corresponding to mass spectrum peaks of the small molecule; and
assigning each of the one or more fragments of the fragmentation graph, a bond type and a log rank.

4. The computer-implemented method of claim 3, wherein the bond type represents chemical bonds that are disconnected in a parent fragment to produce a fragment.

5. The computer-implemented method of claim 3, wherein the log rank represents an intensity of a mass peak corresponding to the fragment.

6. The computer-implemented method of claim 3, wherein the fragmentation graph is generated by performing operations comprising:
cutting, for the small molecule, N—C, O—C, and C—C bonds of the small molecule to generate sub-fragments to form a metabolite graph;
generating depth one fragments by searching the metabolite graph for each cut bond;
determining intersections between each depth one fragment and a corresponding parent fragment;
forming depth two fragments based on the intersection; and
connecting the metabolite graph to each depth one node and each depth two node to a corresponding depth one node.

7. The computer-implemented method of claim 3, wherein assigning the fragmentation graph a log rank comprises:
accessing mass spectra data including a plurality of mass spectra;
for each mass spectra of the plurality:
determining an intensity of each mass peak of the mass spectrum; and
assigning a value for the log rank, wherein the value represents a higher rank as a function of a number of instances of the fragment in the fragmentation graph.

8. The computer-implemented method of claim 1, further comprising:
identifying a drug based on the identified small molecule.

9. The computer-implemented method of claim 1, further comprising:
evaluating an accuracy of the probability that a molecular structure corresponds to the query spectrum by applying a constrained graph variational auto-encoder.

10. The computer-implemented method of claim 1, further comprising:
receiving data representing at least one biosynthetic gene cluster including a non-ribosomal peptide biosynthetic gene cluster, a ribosomally synthesized and posttranslationally modified biosynthetic gene cluster, a polyketide biosynthetic gene cluster, a carbohydrate gene cluster, a polysaccharide gene cluster, or an aminoglycoside gene cluster;
generating a predicted mass spectrum associated with the at least one biosynthetic gene cluster; and
training the machine learning model using the predicted mass spectrum and the at least one biosynthetic gene cluster.

11. The computer-implemented method of claim 1, wherein a p-value associated with the molecular structure and the query spectrum is determined based on a Markov Chain Monte Carlo model.

12. A system for searching a database to identify structures of molecular compounds from mass spectrometry data, the system comprising:
at least one processor; and
a memory storing instructions that, when executed by the at least one processor, cause the at least one processor to perform operations comprising:
receiving a query for a target molecular structure in the database, the query representing a query spectrum;
accessing a machine learning model trained with molecule-spectrum pairs, wherein a molecule-spectrum pair of the molecule-spectrum pairs comprises structure data representing two-dimensional molecular structures of small molecules, the structure data being associated with spectrum data representing a mass spectrum that is generated from the small molecules represented in the structure data;
inputting the query spectrum into the machine learning model;
generating, from the machine learning model, a score for each of one or more molecular structures, each score representing a probability that a molecular structure corresponds to the query spectrum;
selecting, based on each of the scores, a small molecule; and
outputting, on a user interface, a representation of the small molecule.

13. The system of claim 12, wherein the machine learning model enables a reduction in computing memory and a decrease in latency of returning the representation of the small molecule in response to receiving the query spectrum, the reduction being relative to a rule-based model using at least one of bond type data, hydrogen rearrangement data, and dissociation energy data for searching the database.

14. The system of claim 12, the operations further comprising training the machine learning model by performing operations comprising:
generating, for a given small molecule of the structure data, a fragmentation graph, the fragmentation graph representing one or more fragments of the small molecule, the one or more fragments corresponding to mass spectrum peaks of the small molecule; and
assigning each of the one or more fragments of the fragmentation graph, a bond type and a log rank.

15. The system of claim 14, wherein the bond type represents chemical bonds that are disconnected in a parent fragment to produce a fragment.

16. The system of claim 14, wherein the log rank represents an intensity of a mass peak corresponding to the fragment.

17. The system of claim 14, wherein the fragmentation graph is generated by performing operations comprising:
cutting, for the small molecule, N—C, O—C, and C—C bonds of the small molecule to generate sub-fragments to form a metabolite graph;
generating depth one fragments by searching the metabolite graph for each cut bond;
determining intersections between each depth one fragment and a corresponding parent fragment;
forming depth two fragments based on the intersection; and
connecting the metabolite graph to each depth one node and each depth two node to a corresponding depth one node.

18. The system of claim 14, wherein assigning the fragmentation graph a log rank comprises:
accessing mass spectra data including a plurality of mass spectra;
for each mass spectra of the plurality:
determining an intensity of each mass peak of the mass spectrum; and
assigning a value for the log rank, wherein the value represents a higher rank as a function of a number of instances of the fragment in the fragmentation graph.

19. The system of claim 12, the operations further comprising:
evaluating an accuracy of the probability that a molecular structure corresponds to the query spectrum by applying a constrained graph variational auto-encoder.

20. The system of claim 12, the operations further comprising:
receiving data representing at least one biosynthetic gene cluster including a non-ribosomal peptide biosynthetic gene cluster, a ribosomally synthesized and posttranslationally modified biosynthetic gene cluster, a polyketide biosynthetic gene cluster, a carbohydrate gene cluster, a polysaccharide gene cluster, or an aminoglycoside gene cluster;
generating a predicted mass spectrum associated with the at least one biosynthetic gene cluster; and
training the machine learning model using the predicted mass spectrum and the at least one biosynthetic gene cluster.

21. The system of claim 12, wherein a p-value associated with the molecular structure and the query spectrum is determined based on a Markov Chain Monte Carlo model.
